SEQUENCE LISTING (75 sequences)

SEQ ID NO: 1 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 2 - protein, 146 aa, Dermatophagoides pteronyssinus
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 3 - protein, 284 aa, Dermatophagoides pteronyssinus
MEAIKNKMQAMKLEKDNAIDRAEIAEQKARDANLRAEKSEEEVRALQKKIQQIENELDQVQEQLSAANTKLEEKEKALQTAEGDVAALNRRIQLIEEDLERSEERLKIATAKLEEASQSADESERMRKMLEHRSITDEERMEGLENQLKEARMMAEDADRKYDEVARKLAMVEADLERAEERAETGESKIVELEEELRVVGNNLKSLEVSEEKAQQREEAHEQQIRIMTTKLKEAEARAEFAERSVQKLQKEVGRLEDELVHEKEKYKSISDELDQTFAELTGY

SEQ ID NO: 4 - protein, 13 aa, Dermatophagoides pteronyssinus
NAETNACSINGNA

SEQ ID NO: 5 - protein, 13 aa, Dermatophagoides pteronyssinus
SINGNAPAEIDLR

SEQ ID NO: 6 - protein, 13 aa, Dermatophagoides pteronyssinus
SCWAFSGVAATES

SEQ ID NO: 7 - protein, 13 aa, Dermatophagoides pteronyssinus
VAATESAYLAYRN

SEQ ID NO: 8 - protein, 13 aa, Dermatophagoides pteronyssinus
YLAYRNQSLDLAE

SEQ ID NO: 9 - protein, 13 aa, Dermatophagoides pteronyssinus
SLDLAEQELVDCA

SEQ ID NO: 10 - protein, 13 aa, Dermatophagoides pteronyssinus
ELVDCASQHGCHG

SEQ ID NO: 11 - protein, 13 aa, Dermatophagoides pteronyssinus
QHGCHGDTIPRGI

SEQ ID NO: 12 - protein, 13 aa, Dermatophagoides pteronyssinus
TIPRGIEYIQHNG

SEQ ID NO: 13 - protein, 13 aa, Dermatophagoides pteronyssinus
YIQHNGVVQESYY

SEQ ID NO: 14 - protein, 13 aa, Dermatophagoides pteronyssinus
VQESYYRYVAREQ

SEQ ID NO: 15 - protein, 13 aa, Dermatophagoides pteronyssinus
YVAREQSCRRPNA

SEQ ID NO: 16 - protein, 13 aa, Dermatophagoides pteronyssinus
CRRPNAQRFGISN

SEQ ID NO: 17 - protein, 13 aa, Dermatophagoides pteronyssinus
RFGISNYCQIYPP

SEQ ID NO: 18 - protein, 13 aa, Dermatophagoides pteronyssinus
CQIYPPNVNKIRE

SEQ ID NO: 19 - protein, 13 aa, Dermatophagoides pteronyssinus
VNKIREALAQTHS

SEQ ID NO: 20 - protein, 13 aa, Dermatophagoides pteronyssinus
LAQTHSAIAVIIG

SEQ ID NO: 21 - protein, 13 aa, Dermatophagoides pteronyssinus
YHAVNIVGYSNAQ

SEQ ID NO: 22 - protein, 13 aa, Dermatophagoides pteronyssinus
GYSNAQGVDYWIV

SEQ ID NO: 23 - protein, 13 aa, Dermatophagoides pteronyssinus
VDYWIVRNSWDTN

SEQ ID NO: 24 - protein, 13 aa, Dermatophagoides pteronyssinus
NSWDTNWGDNGYG

SEQ ID NO: 25 - protein, 13 aa, Dermatophagoides pteronyssinus
GDNGYGYFAANID

SEQ ID NO: 26 - protein, 13 aa, Dermatophagoides pteronyssinus
FAANIDLMMIEEY

SEQ ID NO: 27 - protein, 12 aa, Dermatophagoides pteronyssinus
MMIEEYPYVVIL

SEQ ID NO: 28 - protein, 13 aa, Dermatophagoides pteronyssinus
ARDQVDVKDCANH

SEQ ID NO: 29 - protein, 13 aa, Dermatophagoides pteronyssinus
KDCANHEIKKVLV

SEQ ID NO: 30 - protein, 13 aa, Dermatophagoides pteronyssinus
IKKVLVPGCHGSE

SEQ ID NO: 31 - protein, 13 aa, Dermatophagoides pteronyssinus
GCHGSEPCIIHRG

SEQ ID NO: 32 - protein, 13 aa, Dermatophagoides pteronyssinus
CIIHRGKPFQLEA

SEQ ID NO: 33 - protein, 13 aa, Dermatophagoides pteronyssinus
PFQLEAVFEANQN

SEQ ID NO: 34 - protein, 13 aa, Dermatophagoides pteronyssinus
FEANQNTKTAKIE

SEQ ID NO: 35 - protein, 13 aa, Dermatophagoides pteronyssinus
KTAKIEIKASIDG

SEQ ID NO: 36 - protein, 13 aa, Dermatophagoides pteronyssinus
KASIDGLEVDVPG

SEQ ID NO: 37 - protein, 13 aa, Dermatophagoides pteronyssinus
EVDVPGIDPNACH

SEQ ID NO: 38 - protein, 13 aa, Dermatophagoides pteronyssinus
DPNACHYMKCPLV

SEQ ID NO: 39 - protein, 13 aa, Dermatophagoides pteronyssinus
MKCPLVKGQQYDI

SEQ ID NO: 40 - protein, 13 aa, Dermatophagoides pteronyssinus
GQQYDIKYTWNVP

SEQ ID NO: 41 - protein, 13 aa, Dermatophagoides pteronyssinus
YTWNVPKIAPKSE

SEQ ID NO: 42 - protein, 13 aa, Dermatophagoides pteronyssinus
VKVMGDDGVLACA

SEQ ID NO: 43 - protein, 13 aa, Dermatophagoides pteronyssinus
GVLACAIATHAKI

SEQ ID NO: 44 - protein, 13 aa, Dermatophagoides pteronyssinus
MEAIKNKMQAMKL

SEQ ID NO: 45 - protein, 13 aa, Dermatophagoides pteronyssinus
MQAMKLEKDNAID

SEQ ID NO: 46 - protein, 13 aa, Dermatophagoides pteronyssinus
KDNAIDRAEIAEQ

SEQ ID NO: 47 - protein, 13 aa, Dermatophagoides pteronyssinus
AEIAEQKARDANL

SEQ ID NO: 48 - protein, 13 aa, Dermatophagoides pteronyssinus
ARDANLRAEKSEE

SEQ ID NO: 49 - protein, 13 aa, Dermatophagoides pteronyssinus
EEVRALQKKIQQI

SEQ ID NO: 50 - protein, 13 aa, Dermatophagoides pteronyssinus
KKIQQIENELDQV

SEQ ID NO: 51 - protein, 14 aa, Dermatophagoides pteronyssinus
ARKLAMVEADLERA

SEQ ID NO: 52 - protein, 14 aa, Dermatophagoides pteronyssinus
EADLERAEERAETG

SEQ ID NO: 53 - protein, 14 aa, Dermatophagoides pteronyssinus
VELEEELRVVGNNL

SEQ ID NO: 54 - protein, 14 aa, Dermatophagoides pteronyssinus
RVVGNNLKSLEVSE

SEQ ID NO: 55 - protein, 13 aa, Dermatophagoides pteronyssinus
EHRSITDEERMEG

SEQ ID NO: 56 - protein, 13 aa, Dermatophagoides pteronyssinus
EERMEGLENQLKE

SEQ ID NO: 57 - protein, 13 aa, Dermatophagoides pteronyssinus
ENQLKEARMMAED

SEQ ID NO: 58 - protein, 13 aa, Dermatophagoides pteronyssinus
RMMAEDADRKYDE

SEQ ID NO: 59 - protein, 13 aa, Dermatophagoides pteronyssinus
KYKSISDELDQTF

SEQ ID NO: 60 - protein, 12 aa, Dermatophagoides pteronyssinus
ELDQTFAELTGY

SEQ ID NO: 61 - protein, 60 aa, Dermatophagoides pteronyssinus
MEAIKNKMQAMKLEKDNAIDRAEIAEQKARDANLRAEKSEEEVRALQKKIQQIENELDQV

SEQ ID NO: 62 - protein, 60 aa, Penaeus aztecus
MDAIKKKMQAMKLEKDNAMDRADTLEQQNKEANNRAEKSEEEVHNLQKRMQQLENDLDQV

SEQ ID NO: 63 - protein, 60 aa, Portunus pelagicus
MDAIKKKMQAMKLEKDDAMDRADTLEQQNKEANIRAEKAEEEVHNLQKRMQQLENDLDQV

SEQ ID NO: 64 - protein, 60 aa, Dermatophagoides pteronyssinus
QEQLSAANTKLEEKEKALQTAEGDVAALNRRIQLIEEDLERSEERLKIATAKLEEASQSA

SEQ ID NO: 65 - protein, 60 aa, Penaeus aztecus
QESLLKANIQLVEKDKALSNAEGEVAALNRRIQLLEEDLERSEERLNTATTKLAEASQAA

SEQ ID NO: 66 - protein, 60 aa, Portunus pelagicus
QESLLKANTQLEEKDKALSNAEGEVAALNRRIQLLEEDLERSEERLNTATTKLAEASQAA

SEQ ID NO: 67 - protein, 60 aa, Dermatophagoides pteronyssinus
DESERMRKMLEHRSITDEERMEGLENQLKEARMMAEDADRKYDEVARKLAMVEADLERAE

SEQ ID NO: 68 - protein, 60 aa, Penaeus aztecus
DESERMRKVLENRSLSDEERMDALENQLKEARFLAEEADRKYDEVARKLAMVEADLERAE

SEQ ID NO: 69 - protein, 60 aa, Portunus pelagicus; identical to SEQ ID NO: 68

SEQ ID NO: 70 - protein, 60 aa, Dermatophagoides pteronyssinus
ERAETGESKIVELEEELRVVGNNLKSLEVSEEKAQQREEAHEQQIRIMTTKLKEAEARAE

SEQ ID NO: 71 - protein, 60 aa, Penaeus aztecus
ERAETGESKIVELEEELRVVGNNLKSLEVSEEKANQREEAYKEQIKTLTNKLKAAEARAE

SEQ ID NO: 72 - protein, 60 aa, Portunus pelagicus; identical to SEQ ID NO: 71

SEQ ID NO: 73 - protein, 44 aa, Dermatophagoides pteronyssinus
FAERSVQKLQKEVGRLEDELVHEKEKYKSISDELDQTFAELTGY

SEQ ID NO: 74 - protein, 44 aa, Penaeus aztecus
FAERSVQKLQKEVDRLEDELVNEKEKYKSITDELDQTFSELSGY

SEQ ID NO: 75 - protein, 44 aa, Portunus pelagicus
FAERSVQKLQKEVDRLEDELVNEKEKYKSTTDELDQAFSELSGY

(19) United States
(12) Patent Application Publication
(10) Pub. No.: US 2017/0153247 A1
(43) Pub. Date: Jun. 1, 2017

(54) METHOD FOR RISK ASSESSMENT OF ALLERGIC REACTION

(71) Applicants: Kang-Hsin Chen, Taipei (TW); Excelsior Bio-System Incorporation, Taipei (TW)

(72) Inventor: Kang-Hsin CHEN, Taipei (TW)

(21) Appl. No.: 15/362,774

(22) Filed: Nov. 28, 2016

Related U.S. Application Data
(60) Provisional application No. 62/260,258, filed on Nov. 26, 2015.

(51) Int. Cl. G01N 33/68; C40B 30/02; G06F 19/22

(52) U.S. Cl. CPC G01N 33/6878; G06F 19/22; C40B 30/02; G01N 2800/24

(57) ABSTRACT

A method for assessing the risk of an individual developing and Immunoglobulin-mediated reaction to one or more allergens increases the specificity of allergy diagnosis and evaluates the specificity of a given allergenic substance. The method may be utilized in in vitro allergy tests, apparatuses, and devices to increase the accuracy and precision of test results. A method to design and to evaluate the effects of personalized peptide molecules for IgE-antigen binding disruption is also presented.

### CROSS-REFERENCE TO RELATED APPLICATIONS

This application claims all benefits accruing under 35 U.S.C. §119 from U.S. provisional application No. 62/260,258 filed on Nov. 26, 2015, the entirety of which is incorporated by reference herein.

### BACKGROUND

1. Field

The present disclosure relates to medical technology and a method for assessing the risk of an individual developing immunoglobulin-mediated allergic reaction to one or more allergenic substances. More particularly, the present disclosure relates to a method to increase the specificity of allergy diagnosis. The present disclosure also relates to a method for evaluating or designing personalized medicine to reduce allergic response of an individual.

2. Background

Immediate hypersensitivity, also named as Type I hypersensitivity, is an allergic reaction mediated by IgE antibodies upon exposure to specific antigens. IgE is a human immunoglobulin secreted by B cell. During Type I hypersensitivity, the specific antigens are recognized by antigen presenting cells, and are presented by T helper cells to stimulate B cell to produce IgE antibodies specific to the particular antigens.

The Fc region of IgE antibody will bind to Fc receptors on mast cells or basophils. When two specific IgEs bind to adjacent Fc receptors on mast cells or basophils, the Fab region of specific IgE may bind to epitopes of the antigen. The epitope is an antigenic region on an antigen and it is the antigenic determinant of the antigen. The epitope is capable of eliciting an immune response when binding to immunoglobulins. The epitope may refer to a peptide, glycan, glycopeptides, or any other patches of molecules that can be bind to immunoglobulins. Two antigen-binding IgE may bind to adjacent Fc receptors, which results in a cross-linking of Fc receptors on mast cell or basophils. The cross-link triggers degranulation of mast cell or basophil. Degranulation is a process of cytoplasmic granules in mast cells, and basophils may release histamine, leukotrienes, prostaglandins, or other allergic mediators.

After the degranulation of mast cells or basophils, the surrounding tissues may be activated by the allergic mediators, causing vasodilation or smooth-muscle contraction. The clinical syndromes of the above immediate hypersensitivity reaction may include but are not limited to: allergic asthma, allergic conjunctivitis, allergic rhinitis, or anaphylaxis.

In vitro allergy testing is often used to evaluate the possibility of immediate hypersensitivity of a subject to certain allergenic substances. The allergenic substances may refer to a group of materials that trigger IgE-mediated hypersensitivities. The allergenic substance may contain one or more allergens. Several in vitro laboratory tests for IgE antibodies have been suggested to assist the diagnosis of Type I hypersensitivity. Common allergen-specific IgE tests for the qualitative or quantitative measurement of IgE levels are Multiple Allergen Simultaneous Test (MAST), Radioallergosorbent Test (RAST) or microarray. In these laboratory tests, specific antigens are used as probes to detect the presence of IgE antibodies in the sample. The detection of IgE antibodies in the above laboratory tests may be enhanced by chemiluminescence, fluorescence, or isotope molecules. A computer-assisted in vitro diagnostic device commercially available for quantitative and qualitative measurement of allergen-specific IgE is, for example, ImmunoCAP developed by Phadia.

Various allergens are utilized for measuring IgE antibodies in in vitro laboratory tests. The allergens can be extracted from allergenic substances derived from natural origins, e.g. dust mites, cockroaches, oranges, shrimps, peanuts, or other common allergens epidemiologically significant. However, a particular allergenic substance in different brands or different batches of the same type of allergen-specific IgE test may contain different antigens or epitopes. This variance could be attributed to differences on extraction protocols, manufacturing procedures, species, time of harvest, or production site of the allergenic substance. Therefore, different substances used in similar tests as the same allergenic substance may lead to different results. It is also possible for two allergenic substances originating from the same source to have different results due to the different extraction protocols or different tissue parts of the source. The inconsistency of antigen-IgE binding among different laboratory tests could lead to inaccurate and imprecise diagnosis.

Recombinant proteins can also be used as a source of materials for allergen-specific IgE tests. Different test kit manufacturers may perform different protocols for manufacturing same-name allergenic substances. Different incubation times, microorganisms, or extraction methods may contribute to different amounts of antigen. Different test kit manufacturers may also use different DNA sequences for particular antigens for same-name allergenic substances. For example, one test kit manufacturer may use recombinant protein of cysteine protease of Dermatophagoides pteronyssinus, or European house dust mite, as the allergen in the allergen-specific IgE test. This allergenic substance in the test may be named “dust mite”. Another test kit manufacturer may also use “dust mite” as one of their allergenic substance in the test, but the other test kit manufacturer may use recombinant protein of cysteine protease of Dermatophagoides microceras, or House dust mite, as the allergen in the allergen-specific IgE test. The two recombinant proteins may be used in laboratory tests and may be named “dust mite”, but they are from different species of dust mites. Therefore, the DNA sequences of above recombinant proteins are different, and may lead to different test results. Abovementioned differences in manufacturing procedure may result in structurally different or quantitatively different antigens in one particular type of allergenic substance in different laboratory tests, thus the types or quantities of epitopes may be affected. This inconsistency may also lead to inaccurate and imprecise diagnosis.

The cross-reactivity of IgE antibodies to allergens may also lead to inaccurate correlation between laboratory test results and clinical symptoms of patients. The cross-reactivity of IgE antibodies to allergens may occur when an epitope has amino acid sequences similar to another epitope on different antigen of the same or another species, and specific IgE antibodies may bind to these epitopes due to their similarity. The cross-reactivity of IgE antibodies to allergen may generate laboratory test results that suggest a subject is allergic to a certain allergen, while the patient does not show clinical symptoms toward the allergenic substance.

Lack of cross-linking of Fc receptors is another reason for inaccurate correlation between laboratory tests and clinical symptoms. The degranulation of mast cells or basophils is triggered by the cross-linking of two or more spatially close and activated Fc receptors. The cross-linking of Fc receptors may be activated by the binding of IgE antibody to two different epitopes on the same antigen, or two similar epitopes being located on different region of the same antigen. The degranulation of mast cells or basophils would require two IgE antibodies, each targeting different epitopes, or the same epitopes located on different region of the same antigen. A positive result may be obtained from laboratory tests to indicate the presence of one particular type of IgE in a serum, but the patient will not show clinical symptoms due to the absence of another type of specific IgE.

IgE antibodies on Fc receptors should be able to bind to two adjacent epitopes to trigger the cross-linking of Fc receptors. The distance between two epitopes on the same protein must be within a given proximity. The allergic reaction cannot be activated when two epitopes on the same protein are too distant from each other.

The cross-linking of IgE Fc receptors on mast cells or basophils would be associated with sufficient amount of IgE antibodies binding to Fc receptors. The limited amount of IgE Fc receptors on mast cells or basophils could only receive a given amount of IgE antibodies. The IgE Fc receptors may be pre-occupied by IgE antibodies specific to one antigen when another antigen is presented. The IgE Fc receptors would not able to accommodate IgE antibodies specific to another antigen, therefore the allergic reaction cannot be triggered.

### BRIEF DESCRIPTION OF THE DRAWINGS

The present disclosure is illustrated by way of embodiments and accompanying drawings.

FIG. 1 is an illustration of the mechanism of IgE-mediated mast cell degranulation in accordance with aspects of the present disclosure.

FIG. 2 is an illustration of IgE-mediated mast cell degranulation triggered by an allergenic substance with more than two antigens in accordance with aspects of the present disclosure.

FIG. 3 is an illustration of IgE-mediated mast cell degranulation triggered by two antigens with two sets of similar epitopes in accordance with aspects of the present disclosure.

FIG. 4 is an illustration of IgE-mediated mast cell degranulation triggered by two antigens with different distributions of epitopes in accordance with aspects of the present disclosure.

FIG. 5 is an illustration of IgE-mediated mast cell degranulation triggered by two antigens with different epitopes in accordance with aspects of the present disclosure.

FIG. 6 is an illustration of IgE-mediated mast cell degranulation triggered by two antigens with different epitopes that have various epitopic distances in accordance with aspects of the present disclosure.

FIG. 7 is an illustration of a database-assisted allergen-specific IgE test for assessing allergenicity of particular allergenic substances.

FIG. 8 is amino acid sequences of allergens of house dust mites. The underlined sections are applied to design overlapping peptides in one embodiment.

FIG. 9A is an illustration of peptide fragments of Der-p1 antigens applied in an exemplary embodiment of a peptide array. FIG. 9B is an illustration of peptide fragments of Der-p2 antigens applied in the embodiment of the peptide array. FIG. 9C is an illustration of peptide fragments of Der-p10 antigens applied in the embodiment of the peptide array.

FIG. 10 is an illustration of comparisons of various peptide fragments of tropomyosin proteins of house dust mites, blue swimmer crab and brown shrimp.

FIG. 11 is an illustration of an exemplary embodiment of peptide probes spotted on a surface of a peptide array.

FIG. 12A is a result of IgE-antigens of crab inhibition experiments. FIG. 12B is a result of IgE-antigens of shrimp inhibition experiments. FIG. 12C is a result of IgE-antigens of dust mite inhibition experiments. FIG. 12D is a result of IgE-antigens of house dust mite inhibition experiments.

### DETAILED DESCRIPTION

Laboratory tests for measuring serum IgE have been widely used to evaluate the serum IgE level of patients subject to allergies. Although the concentration of allergen-specific IgE antibodies in the serum of a patient can be identified via laboratory tests, the resulting allergen-specific IgE concentration may not correlate with the clinical symptoms of the patient, e.g. the presence of a particular type of IgE may not be associated with clinical allergenic substances identified in medical history. The lack of accuracy of allergen-specific IgE tests has been an important issue and challenge in allergy diagnosis.

The description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

The present disclosure is directed to a method for assessing the risk of an individual developing IgE-mediated allergic reaction to one or more allergens. More particularly, the present disclosure is directed to a method to increase the specificity of allergy diagnosis.

The present disclosure is directed to a method to identify the relationships between epitopes, antigens, and allergenic substances. More particularly, the present disclosure is directed to a method to identify the spatial distribution of epitopes on a given antigen, epitopic distances on a given antigen, amount of epitopes on a given antigen, similarities of epitopes on different antigens, and the concentration of antigens in a given allergenic substance. The method may involve methods for presenting the relationships between epitopes, antigens, and allergenic substances.

The present disclosure is directed to a method to evaluate the allergenicity of a substance. The method may include but is not limited to the following parameters: the distribution of epitopes on a given antigen, epitopic distances on a given antigen, amount of epitopes on a given antigen, similarities of epitopes on different antigens, and concentration of antigens in a given allergenic substance. The method may involve mathematical formulations or algorithms to assess risks of having an allergic reaction when exposed to an allergenic substance.

The present disclosure is further directed to a device or apparatus for allergen-specific IgE antibody tests. More particularly, the present disclosure is directed to in vitro laboratory tests that minimize false-positive results and establish positive correlation between test results and clinical symptoms. The in vitro laboratory test may produce meaningful results that can predict or assess the risks of an individual developing allergic reaction for a given allergenic substance.

The present disclosure is further directed to a method to evaluate allergen specificity. More particularly, the present disclosure is directed to a method of using database to evaluate the specificity of a certain allergenic substance. The allergenic substance may be a material used in allergen-specific IgE tests, such as Multiple Allergen Simultaneous Test (MAST), Radioallergosorbent Test (RAST), microarray, or ImmunoCAP.

The present disclosure is further directed to a method to design personalized peptide molecules for IgE interruption of an individual. The personalized peptide molecules can be designed from known epitopes that cause allergic reactions of an individual. More particularly, the present disclosure is directed to a method to design possible peptide molecules to disrupt antigen-IgE complex formation. The method is based on the results from in vitro laboratory tests. The in vitro laboratory tests identify the relationships between epitopes, antigens, and allergenic substances. The in vitro laboratory tests may incorporate programs or algorithms to calculate the relationships between epitopes, antigens, and allergenic substances, thus preventing false positive results. The method is based on the results from the in vitro laboratory tests to identify particular epitopes that will cause IgE-mediated hypersensitivity, to select or design personalized peptide molecules.

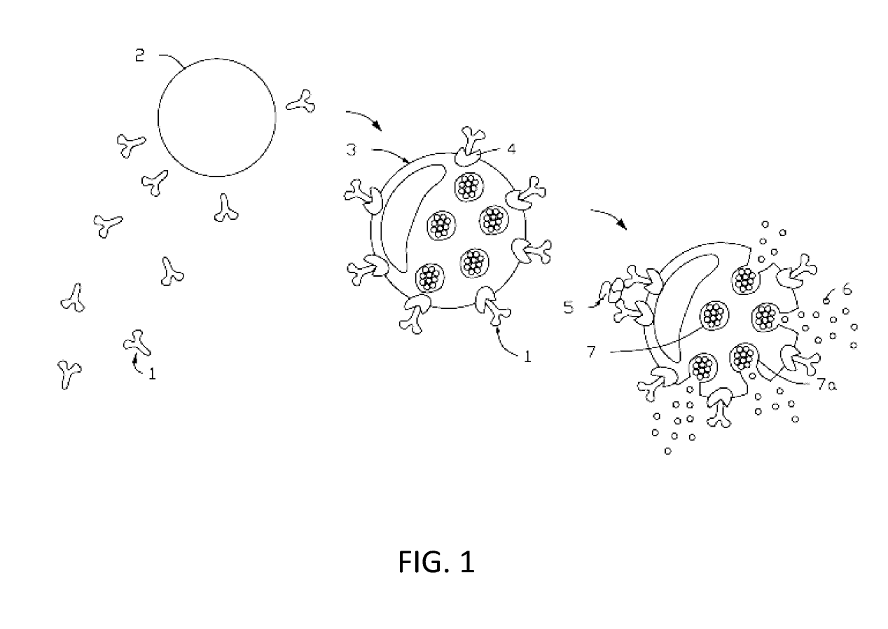

FIG. 1 shows an illustration of IgE-mediated degranulation of mast cells. Activated B-cell 2 will produce IgE antibodies 1. The Fc receptors 4 on the surface of the mast cell 3 have high affinity to the Fc region of IgE. The IgE antibodies 1 may bind to Fc receptors 4. The mast cell 3 contains many cytoplasmic granules 7 within the cytoplasm. When an antigen 5 has been previously recognized by immune system re-ingested by an individual, the antigen-specific IgE antibody 1 that has high affinity with antigen 5 may bind to the epitope 5a of the antigen 5. When two adjacent antigen-specific IgE antibodies 1 on Fc receptors 4 bind to multiple epitopes 5a on one or more antigen 5, cross-linking of Fc receptors 4 will be initiated. The cross-linking of Fe receptors 4 in mast cell 3 will result in the release of immune mediators 6 from degranulated cytoplasmic granules 7a of the mast cells 3, it is this process which may be referred to the degranulation of mast cells. The immune mediators may include but are not limited to: histamine, prostaglandin, and leukotriene. The immune mediators will trigger Type I hypersensitivity by acting directly or indirectly into surrounding smooth muscle tissues.

The interaction between allergens and allergen-specific IgE antibodies may lead to the cross-linking of Fc receptors on mast cells. The present disclosure provides multiple indexes relevant to properties of the allergenic substances, antigens, and epitopes, and the provided multiple indexes are attributable to the tendency of developing Fc receptors cross-linking on the mast cells or basophils.

One approach of the present disclosure is directed to a method for representing the relative amount of different antigens presented in a given allergenic substance. An allergenic substance is a substance that could induce allergic reaction when ingested by an individual. Common allergenic substances include but are not limited to: dust, pollen, mite feces, or latex. The allergenic substances can comprise proteins such as antigens or allergens. An antigen or allergen may be referred to as a protein of the allergenic substance that has high affinity with allergen-specific IgE antibodies. The antigen or an allergen may be also referred as a core element to activate allergic reactions when an allergenic substance is ingested by an individual. A partition index of an allergenic substance is used to represent the relative amounts of different antigens or allergens in a given allergenic substance. The partition index represents the variety of antigens or allergens in a given allergenic substance. When comparing different types of antigens or allergens in a given allergenic substances, the partition index may be used to evaluate the relative immunogenicity of antigens in the given allergenic substance.

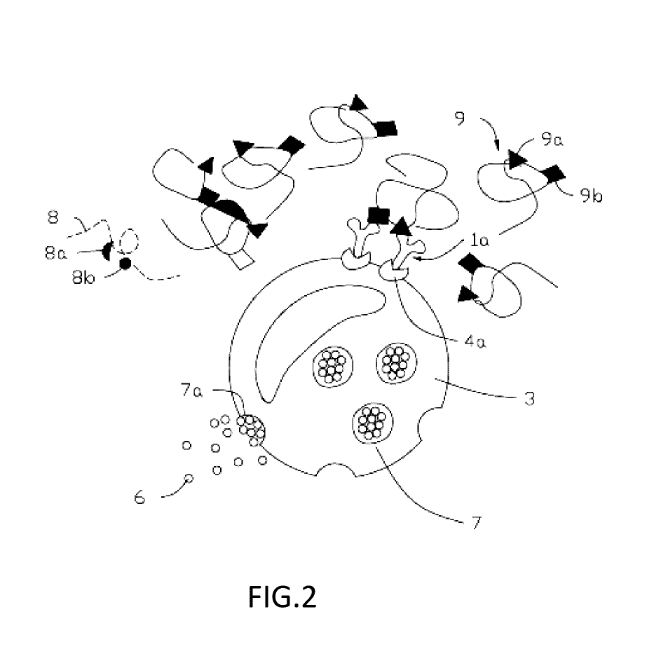

FIG. 2 is an illustration of an allergen activated mast cell degranulation. An allergenic substance comprises two types of antigens, antigen 8 and antigen 9. In the allergenic substance, the concentration of the antigen 8 is lower than the concentration of the antigen 9, as there is only one antigen 8 molecule but multiple antigen 9 molecules in FIG. 2. An IgE antibody 1a is specific to a epitope 9a and a epitope 9b on the antigen 9. The IgE antibody 1a binds to Fc receptors 4a on the mast cell 3, causing the cross-linking of Fc receptors 4a and the degranulation of the mast cell 3. The partition index is relevant to the relative concentrations of antigen 8 and antigen 9 in the same allergenic substance

The partition index is calculated based on an quantification of different antigens in a given allergenic substance. To identify the amount of different antigens in the given allergenic substance, both qualitative and quantitative protein analysis are required, including but not limited to: 2-D protein electrophoresis, high performance liquid chromatography (HPLC), gas chromatography (GC), or MALDI-TOF mass spectrometry analysis.

Considering that false-positive results of allergen-specific IgE tests may be caused by the cross-reactivity of IgE antibodies to allergens, the present disclosure provides a method to represent similarities between epitopes on different antigens. As described previously; the epitope is the antigenic determinant of an antigen. The epitope may be referred to a region of the antigen that binds to Fab region of an IgE antibody. The epitope could be peptide molecules of a region of a protein antigen. One antigen may have many identical or different epitopes while one particular type epitope may also appear on different antigens. Herein, an evolution index is used to represent similarities between epitopes on different antigens. The evolution index may be applied to evaluate the possibility of developing cross-reactivity from epitopes on different antigens. The cross-reactivity may occur when two identical or similar epitopes are presented by different antigens. The cross-reactivity of allergens may occur between closely related species.

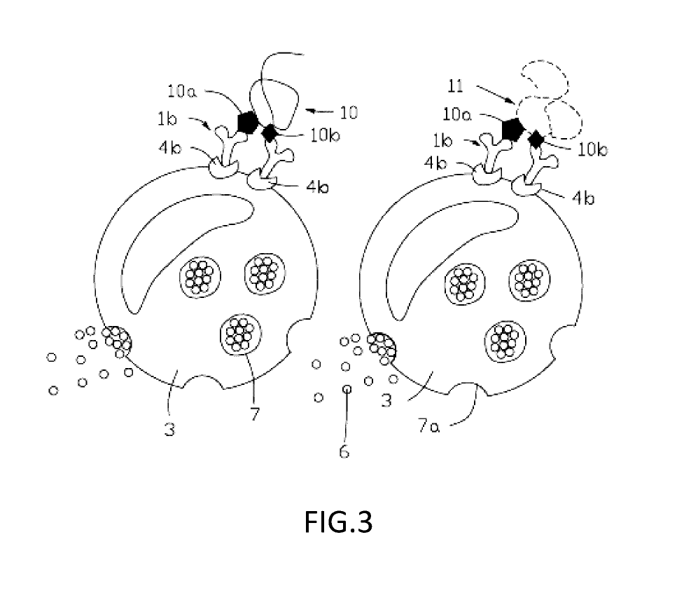

FIG. 3 is another illustration of an allergen activated mast cell degranulation. The IgE antibody 1b is specific to both epitope 10a and epitope 10b. Both the epitope 10a and the epitope 10b are located on antigen 10 and antigen 11. The antigen 10 and the antigen 11 may originate from the same allergenic substance, or may originate from different allergenic substances. In such a case, the evolution index is relevant to the presence of epitope 10a and epitope 10b on both antigen 10 and antigen 11. The evolution index represents how epitope 10a and epitope 10b are presented in different antigens. In conventional allergen-specific IgE tests, the cross-reactivity of IgE antibody to antigen 10 and antigen 11 could occur when only the antigen 10 or only the antigen 11 is used in the laboratory test. The individual who takes the laboratory test may be in fact allergic to only antigen 11 but the conventional laboratory test may demonstrate a positive result on antigen 10. Therefore, the evolution index could serve as a reference to predict the possibility of false positive results caused by cross-reactivity of IgE antibody to antigen 11 and antigen 10.

The evolution index is calculated based on peptide sequences of similar or identical epitopes on different antigens. The peptide sequences on common epitopes can be identified from commercially available epitope databases. Then the evolution index can be generated via a comparison of peptide sequences among different epitopes.

Another factor affecting the binding between IgE and antigen is the amount of epitopes presented on one antigen. One approach of the present disclosure is directed to a method for representing the relative amounts of epitopes on a given antigen. A proportion index of an antigen is used to represent a relative amount of epitopes on a given antigen. The amounts of epitopes that can be recognized and bound to corresponding IgE antibodies varies in different antigens.

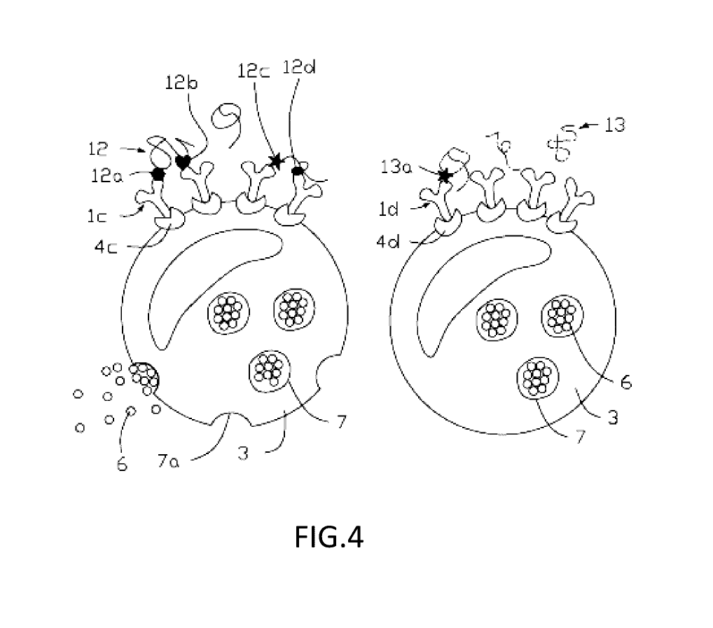

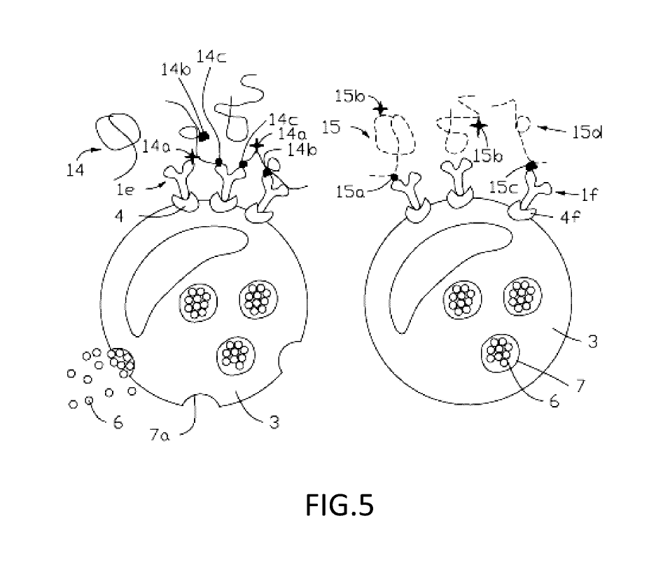

FIG. 4 is another illustration of an allergen activated mast cell degranulation. Epitopes 12a, 12b, 12c, and 12d are located on antigen 12, but only one epitope 13a is located on antigen 13. Although the amount of antigen 13 in FIG. 4 and the amount of antigen 12 in FIG. 4 are the same, the degranulation of the mast cell is more likely to be activated by antigen 12. Due to the antigen 12 having more epitopes, the IgE antibodies 1c on adjacent Fc receptors 4c of the mast cell 3 may have higher tendencies to bind to the antigen 12, leading to a higher cross-linking tendency of the Fc receptors. The proportion index of antigen 12 and antigen 13 are relevant to the tendencies of developing Fc receptor cross-linking when antigen-specific IgEs are present.

The proportion index is calculated based on the amount of epitopes on a given antigen. As antigens triggering allergic reactions have been identified, the region for IgE antibody binding on a given antigen can be recognized by computer-assisted protein modeling or any other bioinformatic tools. Therefore, the amount of possible epitopes on a certain antigen can be determined.

Another factor affecting the binding between IgE and antigen is the relative distance between two epitopes. Two epitopes must be spatially close for the binding of two IgE antibodies. One approach of the present disclosure is directed to a method for representing the relative distribution of epitopes on different antigens of a given allergenic substance. A distribution index of an antigen is used to represent the relative distribution of epitopes on different antigens within a given allergenic substance.

FIG. 5 is another illustration of an allergen activated mast cell degranulation. Antigen 14 and antigen 15 are two antigens of the same allergenic substance. Epitopes 14a, 14b and 14c are located on the antigen 14. Epitopes 15a, 15b, and 15c are located on the antigen 15. The distribution index of the antigen 14 and the antigen 15 represents the distribution of epitopes of different antigens within the same allergenic substance. The distribution index of antigen 14 and antigen 15 are relevant to the tendencies of developing Fc receptor cross-linking when antigen-specific IgEs are present.

The distribution index is calculated based on the antigen information of allergenic substance and the epitope information of antigens. The antigens of a certain allergenic substance can be identified from qualitative protein analysis, which may include but is not limited to: 2-D protein electrophoresis, HPLC, GC, or MALDI-TOF mass spectrometry analysis. Epitopes on a given protein can be identified by protein microarrays, immunoassays, or other qualitative peptide analyses such as liquid chromatography-mass spectrometry (LC-MS). Then, the information retrieved from above analyses can be organized by computer-assisted protein modeling or any other bioinformatic tools. The distribution index can be calculated based on the number of epitopes on an antigen, and the number of different antigens in an allergenic substance.

Another approach of the present disclosure is directed to a method for representing the definitive distance between epitopes on the same antigen. It would require two adjacent and epitope-binding IgE antibodies to activate the cross-linking of Fc receptors. If the antigen-binding IgE antibodies are specific to epitopes on the same antigen molecule, the epitope would needs to be located closely on the antigen molecule. A spatial index of epitopes is used to represent the definitive distance between two epitopes, that is, the spatial relationship of two epitopes on the same antigen.

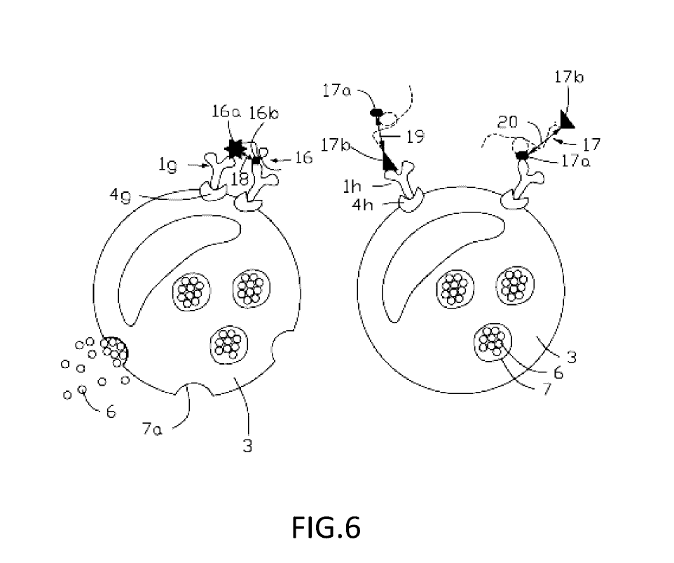

FIG. 6 is another illustration of an allergen activated mast cell degranulation. The epitopic distance may be referred to the distance between two epitopes on one antigen. An epitopic distance 18 between epitope 16a and epitope 16b on antigen 16 is shorter than an epitopic distance 19 between epitope 17a and 17b on antigen 17. The antigen 16 is binding to two separated and adjacent IgE antibodies 1g. Therefore, the Fc receptor cross-linking on mast cell is more likely to be triggered by the binding between the IgE 1g and the antigen 16 because the epitopes 16a and 16b on the antigen 16 are closer when compared with epitope 17a and 17b on antigen 17.

The spatial index is calculated based on the epitope information of a certain antigen. Epitopes on the same antigen can be identified by protein microarrays, immunoassays, or other qualitative peptide analyses such as liquid chromatography-mass spectrometry (LC-MS). The location of epitopes on an antigen can be generated by combining qualitative peptide analyses and computer-assisted protein modeling. The spatial index can be calculated based on the relative distance between any two epitopes of the same antigen.

Another approach of the present disclosure is directed to a method to evaluate the allergenicity of an allergenic substance. The allergenicity may represent the substance's relative possibility to trigger allergy in an individual. The following factors in an allergenic substance can be attributable to the method: the epitopic distances of epitopes on an antigen, the distribution of epitopes on an allergenic substance, the amount of epitopes on an antigen, the similarities between two epitopes on different antigens, and the species of antigens in an allergenic substance. Particular parameters may be used for the method: the spatial index, the distribution index the proportion index, the evolution index, and the partition index. The method may present the relative possibility for an individual to show allergic reaction in response to a given substance. The method can be represented by the following formulation:

The allergenicity of a given allergenic substance=the protein or antigen quantity of the allergenic substance*amount of epitopes*(the spatial index*the distribution index*the proportion index*the evolution index*the partition index).

The spatial index, the distribution index, the proportion index, the evolution index, and the partition index are indexes relevant to epitopes, antigens, and allergenic substances as described above. The information required to generate these indexes can be calculated from qualitative or quantitative protein analyses on a given antigen or allergen. For a particular antigen, the spatial index is relevant to epitopic distances of epitopes on an antigen, and the proportion index is relevant to the amount of epitopes on an antigen. For a particular allergenic substance, the distribution index is relevant to the relative distribution of epitopes on different antigens within an allergenic substance, and the partition index is relevant to the relative amount of different antigens in a given allergenic substance. To evaluate the cross-reactivity of epitopes, the evolution index is relevant to similarities between epitopes on different antigens.

The above formulation represents the interaction between various determinants affecting IgE-mediated allergic response. The above formulation is a mathematical presentation of the allergenicity evaluation. The indexes in the above formulation are derived from the accumulation from results of qualitative and quantitative protein analyses. The method could be used to estimate or evaluate the probability of an individual having an allergic reaction toward certain allergenic substance. The method may generate assessments of risks from laboratory allergy tests. The method may reduce the necessity for an individual to undergo in-vivo allergy tests, thereby reducing the chance of having side effects from the in-vivo allergy tests.

The method may also serve as a method to improve specificity of allergen-specific IgE tests. A positive result from allergen-specific IgE tests may represent the cross-reactivity between epitopes on different species. By utilizing the method, a false positive result can be identified. Additionally, a negative result from allergen-specific IgE test can be false-negative due to the quality of antigens used in the laboratory test.

One approach of the present disclosure is directed to a method to evaluate the antigenicity of a particular antigen. When specific antigens in an allergenic substance have been identified, the antigen information can be a reference for assessing the risk of having allergic reaction toward the antigens. The following factors in an antigen can be attributable to the method: the epitopic distances of epitopes on an antigen and the amount of epitopes on an antigen. The spatial index is relevant to epitopic distances of epitopes on an antigen, and the proportion index is relevant to the amount of epitopes on an antigen. Above indexes are parameters that may be utilized for the method. The method may represent the relevancy of above indexes to the relative possibility for an individual to have allergic reaction to a given antigen. The method can be represented by the following formulation:

The risk of having allergic reaction to a given antigen=the spatial index*the proportion index.

The above formulation is a mathematical presentation of the method. The indexes in the above formulation are derived from the accumulation from results of qualitative and quantitative protein analyses for a particular antigen. The method could be used to estimate or evaluate the possibility for an individual to have allergic reaction toward one certain antigen. When combined with computing devices and diagnostic tests, the method may generate assessments of risks from laboratory allergy tests to assist the research or diagnosis of IgE-mediated hypersensitivity triggered by a given antigen.

One approach of the present disclosure is directed to a method for constructing databases. The database is used to store information of epitopes, antigens, or/and allergenic substances. Such information can be collected from the following sources: immunoassays, protein microarrays, HPLC, MALDI-TOF mass spectrometry, or other protein qualitative or quantitative analyses. Any allergen that has been proven to cause allergy can be the subject of the above protein analyses. Other relevant information may include but is not limited to: the species of an allergenic substance, origin of the substance (location and date of sampling), experiment protocols of the analysis, and the prevalence of allergic reaction triggered by the allergenic substance within a given population. Above information can be input to the database. The database may provide references for allergy diagnosis, or combine the database with any current allergen-specific IgE tests.

One approach of the present disclosure is directed to a method of the use of allergenicity algorithm in diagnostic device or apparatus. Common allergenic substances are often used as solid phases in allergen-specific IgE tests. However, the sources of allergenic substances may come from different materials. Even if the name of one allergenic substance is the same in two different laboratory tests, the sources of allergenic substance can be varied, causing varied results. For example, the allergenic substance named “shrimp” in different laboratory tests may be extracted from different species of shrimps, thus the tests may show up with different results to an individual's specific IgE to shrimp.

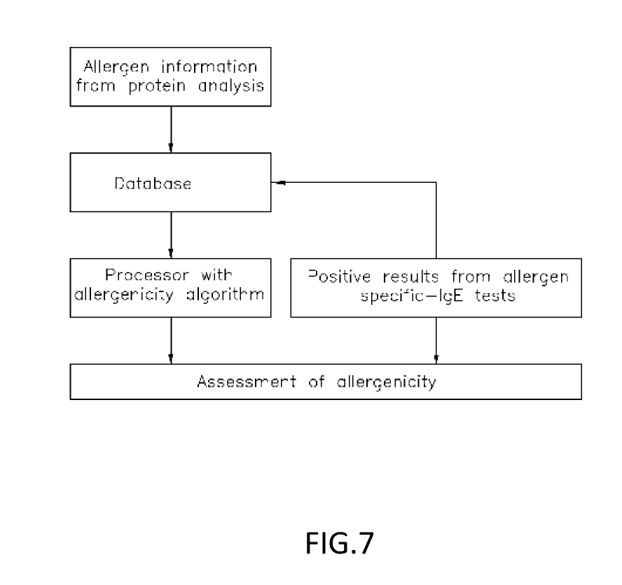

FIG. 7 is an illustration of the use of the database in a given diagnostic device or apparatus. The allergenic substance that would be used in the laboratory tests are first extracted and purified from the source of the material. The material can be biological samples of organisms or inorganics, e.g. latex, pollen, feces of dust mites, tissue samples of German cockroaches, etc. The information of the allergenic substances can be analyzed from protein analyses, the protein analyses may include but are not limited to: 2-D protein electrophoresis, HPLC, GC, or MALDI-TOF mass spectrometry analysis. The information of the allergenic substance from these protein analyses may include but is not limited to: amount of proteins, species of antigens, peptide sequence of antigens, and epitopes of antigens.

The information of particular allergenic substances is then collected from protein or peptide analysis and is stored in the database. Additional information of the allergenic substances will be also added in the database. The additional information may include but is not limited to: the species of an allergenic substance, origin of the material (location and date of sampling), experiment protocols of the analysis, and the prevalence of allergic reaction triggered by the allergenic substance within a given population. The database may be stored in at least one memory device or storing device communicatively linked to the diagnostic device or apparatus, or in existing memory unit within the diagnostic device. The information of allergenic substance may be linked with the same allergenic substance used in laboratory tests, so that a particular allergenic substance in the test can be linked to one or more pieces of information in the database.

The information of one or more particular allergenic substances is then inputted in one or more processors. The processors or computing units are communicatively linked to the diagnostic device or apparatus, or are existing computing unit within the diagnostic device. The processor is utilized with an algorithm. The algorithm may refer to the following mathematical formulation:

The allergenicity of an allergenic substance=the protein or antigen quantity of the allergenic substance*amount of epitopes*(the spatial index*the distribution index*the proportion index*the evolution index*the partition index).

The spatial index, the distribution index, the proportion index, the evolution index, and the partition index are indexes relevant to epitopes, antigens, and allergenic substances. The information required to generate above indexes can be calculated from qualitative or quantitative protein analyses on a given antigen or allergenic. For a particular antigen, the spatial index is relevant to epitopic distances of epitopes on an antigen, and the proportion index is relevant to the amount of epitopes on an antigen. For a particular allergenic substance, the distribution index is relevant to the relative distribution of epitopes on different antigens within an allergenic substance, and the partition index is relevant to the relative amount of different antigens in a given allergenic substance. To evaluate the cross-reactivity of epitopes, the evolution index is relevant to similarities between epitopes on different antigens.

The serum of an individual is collected from blood samples. The serum is then provided to the laboratory tests to analyze the concentration or species of allergen-specific IgE antibodies. The laboratory test may include but is not limited to: Multiple Allergen Simultaneous Test (MAST), Radioallergosorbent Test (RAST), microarray, ImmunoCAP, or any other immunoassays measuring the amount of IgE antibodies in a serum. Once positive results are generated from the laboratory tests, the concentration of particular types of allergen-specific IgE will be identified. Information for the allergenic substance will be fed to the algorithm of the processor. The processor will generate an assessment of allergenicity toward a particular allergenic substance. The assessment will combine the results of the allergen-specific IgE concentration from the test, and the allergenic substance information from the database. The assessment assists the diagnosis of allergy. The above procedure may increase the accuracy and precision of allergen-specific IgE tests, and provides positive correlation between laboratory tests and clinical history of an individual.

Another approach of the present disclosure is directed to a method of using database to evaluate the specificity of a certain allergenic substance. More particularly, the present disclosure is directed to a method to evaluate the possibility of an allergenic substance developing cross-reactivity in relation to two or more types of IgE antibodies. The allergenic substance may be a material used in allergen-specific IgE tests for the purpose of measuring specific IgE levels.

The allergenic substance may be extracted from natural origins, such as biological samples or inorganics. The allergenic substance may also comprise one or more recombinant proteins. The information of the allergenic substance will be analyzed by qualitative or quantitative protein analyses. The protein analyses may include but are not limited to: 2-D protein electrophoresis, HPLC, GC, or MALDI-TOF mass spectrometry analysis. The information of the allergenic substance from these protein analyses may include but not limited to: amount of proteins, species of antigens, peptide sequence of antigens, and epitopes of antigens. Above information can be constructed into a database. The information of allergenic substances used in allergen-specific IgE tests, antigens in the allergenic substances, and epitopes in the antigens are collected into the database.

The information of allergenic substances is then processed by a computing device or processor. The processor is communicatively linked to the diagnostic device or apparatus, or there can be an existing computing unit within the diagnostic device. An algorithm is provided and inputted into the device or processor, a mathematical formulation of the algorithm may be refer to:

The possibility of cross-reactivity of an allergenic substance=the protein or antigen quantity of the allergenic substance*amount of epitopes*(the spatial index*the distribution index*the proportion index*the evolution index*the partition index.

The spatial index, the distribution index, the proportion index, the evolution index, and the partition index are indexes relevant to epitopes, antigens, and allergenic substances. The information required to generate above indexes can be calculated from qualitative or quantitative protein analyses on a given antigen or allergen. For a particular antigen, the spatial index is relevant to distances between epitopes on an antigen, and the proportion index is relevant to the amount of epitopes on an antigen. For a particular allergenic substance, the distribution index is relevant to the relative distribution of epitopes on different antigens within an allergenic substance, and the partition index is relevant to the relative amount of different antigens in a given allergenic substance. To evaluate the cross-reactivity of epitopes, the evolution index is relevant to similarities between epitopes on different antigens.

With the algorithm, the information of the allergenic substance is re-organized into the form of parameters for calculating above indexes. The algorithm may generate an assessment representing the specificity of the allergenic substance. The assessment may be a reference for selecting allergenic substances when designing or manufacturing allergen-specific IgE tests. The allergenic substance with the lowest possibility of developing cross-reactivity is preferred in the laboratory tests, because false-positive results are less likely to take place. The specificity of each allergenic substance is evaluated to increase the overall specificity of allergy testing.

Another approach of the present disclosure is directed to a method to design and to evaluate the therapeutic effects of personalized peptide molecules for IgE-antigen binding disruption. The epitopes for binding of allergen-specific IgE antibodies are identified by laboratory tests combining the database for allergic reactions. The epitope information may include but is not limited to: the amount of epitopes on an antigen, the peptide sequences of epitopes, and the positions of epitopes on an antigen. Based on the epitope information, several peptide compounds that mimic an allergenic epitope can be synthesized and block the IgE-antigen binding when the synthesized peptide compounds are delivered to the patient. Therefore, the IgE-antigen binding may be disrupted by the blocking, and the symptoms of IgE-mediated hypersensitivity can be reduced by the peptide.

The therapeutic effect of the synthesized peptide compound(s) can be evaluated by the following mathematical formulation:

The therapeutic effects of blocking IgE-antigen binding by synthesized peptide compound=types of peptide compounds*delivery dosage of a particular peptide compound*evolution index*.

A group of several similar peptide compounds with different peptide sequences may be produced for blocking a known IgE-antigen binding in the patient. The synthesized peptide compounds may be similar in peptide sequences but may have different therapeutic effects. The delivery dosage of a particular peptide compound in the group of peptide compounds is given for evaluating the optimal therapeutic dosage of a particular peptide compound. For reducing the possibility of cross-reactivity between different antigens, the evolution index is included in the formulation. The evolution index represents the similarities of epitopes on different antigens. The evolution index may be used to evaluate the possibility of developing cross-reactivity from epitopes on different antigens. The evolution index can be acquired from the database provided in the example of FIG. 7 for assessing allergenicity.

The above mathematical formulation is used to determine the IgE-antigen binding blocking effect of a particular peptide compound before actual delivery. The peptide compound with the greatest ability to block the IgE-antigen binding can be identified by the formulation and be delivered in an optimized dosage for a patient. The mathematical formulation may greatly reduce the cost of drug development, and provide a solution for personalized medicines on allergy treatment.

An exemplary embodiment of the methods disclosed in previous paragraphs is described below.

1. Peptide Array Design.

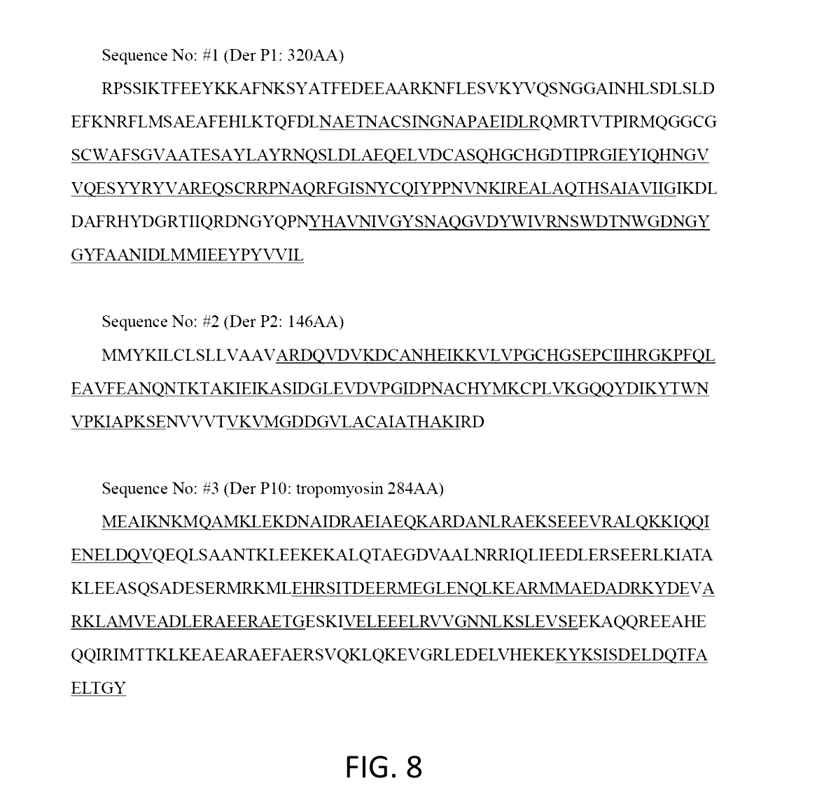

Der p1, Der p2, and Der p10 are antigens of house dust mites, and their amino acid sequences can be obtained from allergenic databases (for example, the WHO/IUIS Allergen Database). Their amino acid sequences (Sequence No. #1, #2, #3) are presented in FIG. 8 and the underlined parts of each sequence could be used as probes of a peptide array, which is selected based on Chapman (1980). Heymnann, Chapman, Aalberse and Fox (1989) and Asturias et al. (1998). To investigate peptide epitopes that bind to specific IgE in a serum, the underlined amino acid sequences could be further sectioned to overlapping peptide fragment libraries to improve the specificity. The length of each overlapping peptide fragment could be from 10-15 amino acids. Table 1

TABLE 1
Peptide fragments of Der p1 antigen

| Seq | | Amino | Seq | | Amino |
|---|---|---|---|---|---|
| No. | No. | acid sequence | No. | No. | acid sequence |
| #4 | Der p1-1 | NAETNACSINGNA | #16 | Der p1-13 | CRRPNAQRFGISN |
| #5 | Der p1-2 | SINGNAPAEIDLR | #17 | Der p1-14 | RFGISNYCQIYPP |
| #6 | Der p1-3 | SCWAFSGVAATES | #18 | Der p1-15 | CQIYPPNVNKIRE |
| #7 | Der p1-4 | VAATESAYLAYRN | #19 | Der p1-16 | VNKIREALAQTHS |
| #8 | Der p1-5 | YLAYRNQSLDLAE | #20 | Der p1-17 | LAQTHSAIAVIIG |
| #9 | Der p1-6 | SLDLAEQELVDCA | #21 | Der p1-18 | YHAVNIVGYSNAQ |
| #10 | Der p1-7 | ELVDCASQHGCHG | #22 | Der p1-19 | GYSNAQGVDYWIV |
| #11 | Der p1-8 | QHGCHGDTIPRGI | #23 | Der p1-20 | VDYWIVRNSWDTN |
| #12 | Der p1-9 | TIPRGIEYIQHNG | #24 | Der p1-21 | NSWDTNWGDNGYG |
| #13 | Der p1-10 | YIQHNGVVQESYY | #25 | Der p1-22 | GDNGYGYFAANID |
| #14 | Der p1-11 | VQESYYRYVAEEQ | #26 | Der p1-23 | FAANIDLMMIEEY |
| #15 | Der p1-12 | YVAREQSCRRPNA | #27 | Der p1-24 | MMIEEYPYVVIL |

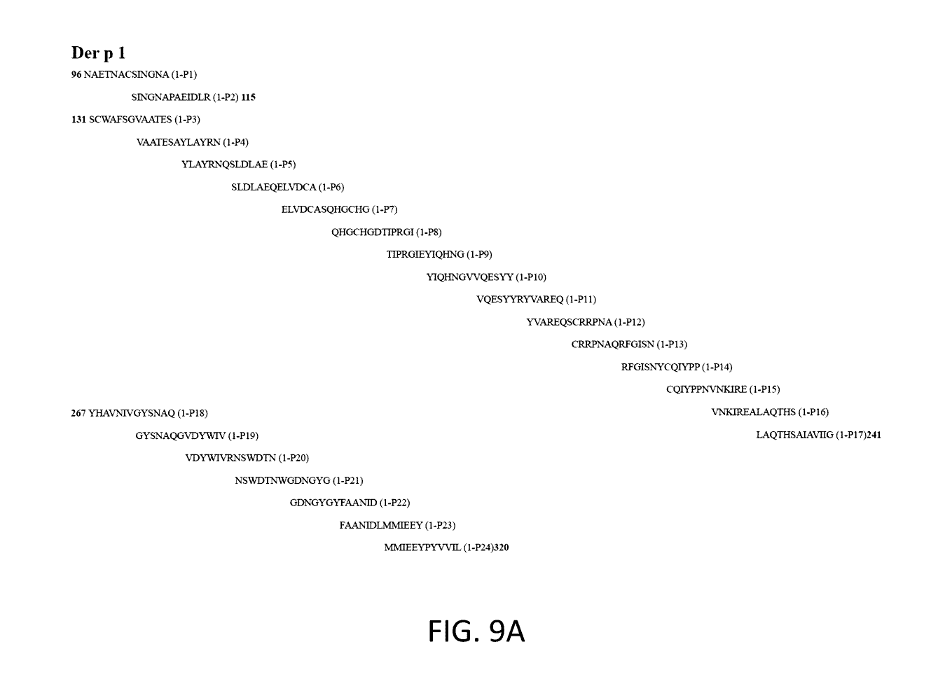

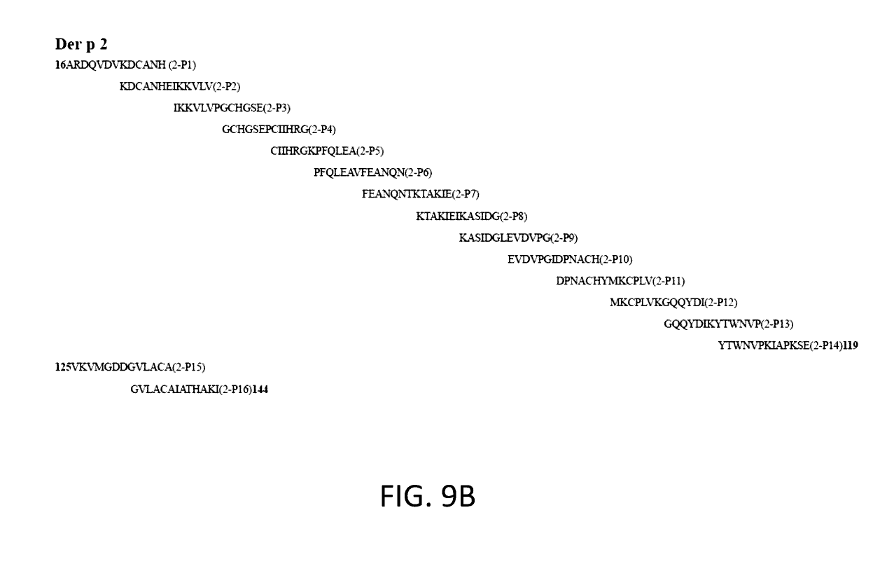

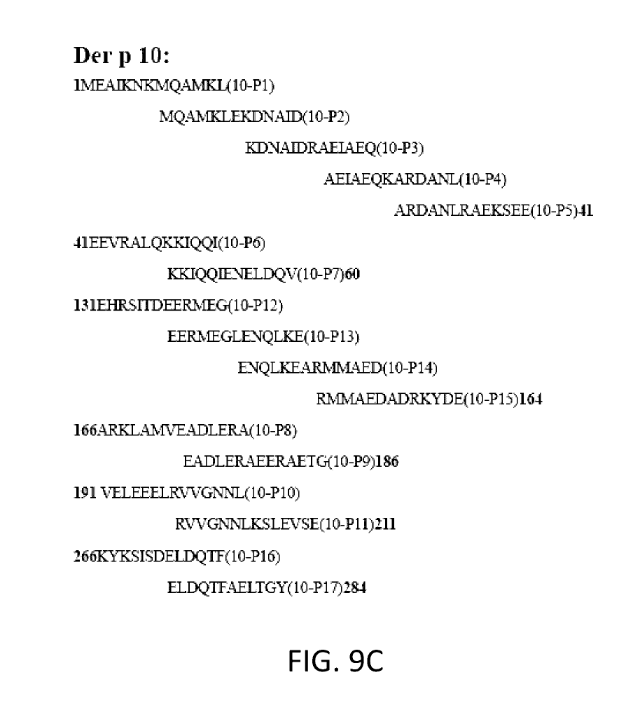

shows the overlapping peptide fragment library originating from Der p1 antigen designed according to the present embodiment (Sequence No. #4-#27). Table 2 shows the overlapping peptide fragment library originating from Der p2 antigen designed according to the present embodiment (Sequence No. #28-#43). Table 3 shows the overlapping peptide fragment library originating from Der p10 antigen designed according to the present embodiment (Sequence No. #44-#60). FIGS. 9A. 9B and 9C present the original position of each peptide fragment in the corresponding antigen, wherein numbers labeled before or after one peptide fragment indicate the position of it in the full peptide sequence of the corresponding antigen.

TABLE 2
Peptide fragments of Der p2 antigen

| Seq | | Amino | Seq | | Amino |
|---|---|---|---|---|---|
| No. | No. | acid sequence | No. | No. | acid sequence |
| #28 | Der p2-1 | ARDQVDVKDCANH | #316 | Der p2-13 | KASIDGLEVDVPG |
| #29 | Der p2-2 | KDCANHEIKKVLV | #37 | Der p2-14 | EVDVPGIDPNACH |
| #306 | Der p2-3 | IKKLVLPGCHGSE | #38 | Der p2-15 | DPNACHYMKCPLV |
| #31 | Der p2-4 | GCHGSEPCIIHRG | #39 | Der p2-16 | MKCPLVKGQQYDI |
| #32 | Der p2-5 | CIIHRGKPFQLEA | #420 | Der p2-17 | GQQYDIKYTWNVP |
| #339 | Der p2-6 | PFQLEAVFEANQN | #41 | Der p2-18 | YTWNVPKIAPKSE |
| #34 | Der p2-7 | FEANQNTKTAKIE | #4 | Der p2-19 | VKVMGDDGVLACA |
| #35 | Der p2-8 | KTAKIEIKASIDG | #43 | Der p2-20 | GVLACAIATHAKI |

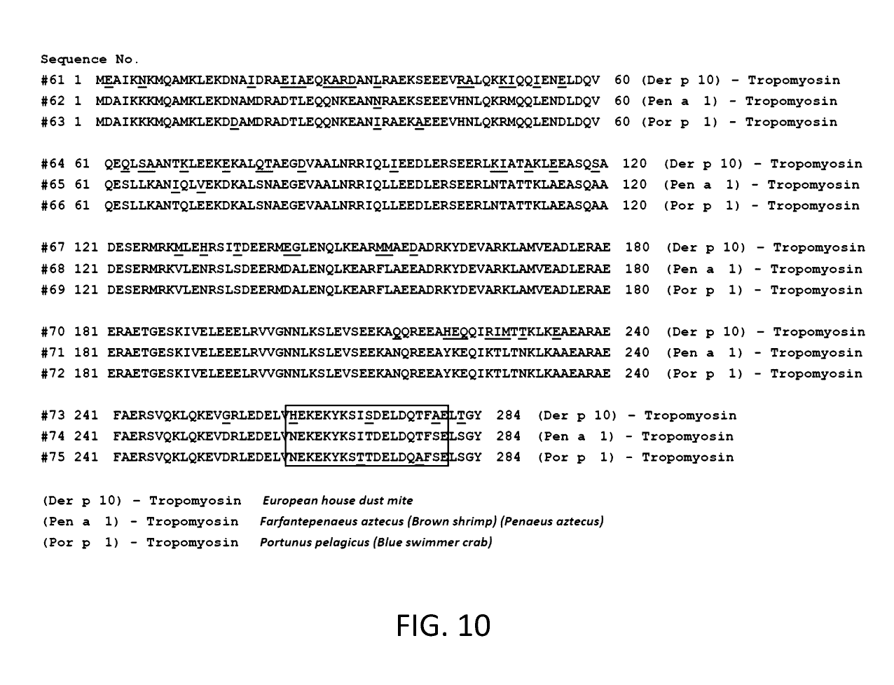

FIG. 10 shows a comparison of various peptide fragments of tropomyosin proteins of house dust mite (L)Dermatophagoides pteronyssinus), blue swimmer crabs (Portunus pelagicus) and brown shrimp (Penaeus aztecus). Compared to Por P1 antigen of crabs (#63. #66, #69, #72, #75). Pit v1 and Pen a2 antigens of shrimps ((#62, #65, #68, #71, #74), it could be found that amino acid sequences of Der p10 (#61, #64, #67, #70, #73) show relatively high percentage of homology with Por P1 antigen of crabs, Pit v1 and Pen a2 antigens of shrimps. The amino acid sequences highlighted in box in FIG. 10 contain the sequences of No. Der p10-16 (#59) and No. Der p10-17 (#60) (presented in Table 3).

TABLE 3
Peptide fragments of Der p10 antigen

| Seq | | Amino | Seq | | Amino |
|---|---|---|---|---|---|
| No. | No. | acid sequence | No. | No. | acid sequence |
| #44 | Der p10-1 | MEAIKNKMQAMKL | #53 | Der p10-10 | VELEEELRVVGNNL |
| #45 | Der p10-2 | MQAMKLEKDNAID | #54 | Der p10-11 | RVVGNNLKSLEVSE |
| #46 | Der p10-3 | KDNAIDRAEIAEQ | #55 | Der p10-12 | EHRSITDEERMEG |
| #47 | Der p10-4 | AEIAEQKARDANL | #56 | Der p10-13 | EERMEGLENQLKE |
| #48 | Der p10-5 | ARDANLRAEKSEE | #57 | Der p10-14 | ENQLKEARMMAED |
| #49 | Der p10-6 | EEVRALQKKIQQI | #58 | Der p10-15 | RMMAEDADRKYDE |
| #50 | Der p10-7 | KKIQQIENELDQV | #59 | Der p10-16 | KYKSISDELDQTF |
| #51 | Der p10-8 | ARKLAMVEADLERA | #60 | Der p10-27 | ELDQTFAELTGY |
| #52 | Der p10-9 | EADLERAEERAETG | | | |

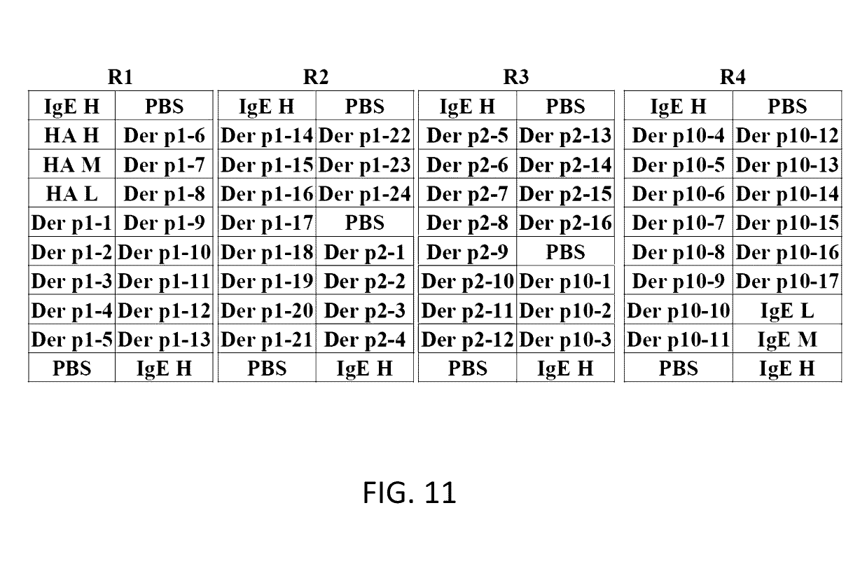

A plurality of peptide probes are synthesized according to each of the above peptide fragments of Der p1, Der p2, and Der p10. Theses synthesized peptide probes are spotted on a solid surface to construct a peptide array. The peptide array includes at least one reaction area, and each reaction area includes at least one reaction block. FIG. 11 shows an example of the arrangement of peptide probes spotted on each of the blocks at the surface of the peptide array, wherein each of the peptide fragments of Der p1, Der p2, and Der p10 is spotted twice. The peptide array of FIG. 11 includes at least one reaction area, and each reaction area includes 4 reaction blocks, namely R1, R2, R3, and R4.

2. Serum Samples Analysis Tests.

Serum samples are used and labeled to evaluate the peptide array of the present embodiment. The labeled serum samples include 23 positive serum samples obtained from patients showing allergy symptoms, one negative serum sample, and one blank. Conventional protein arrays are also used in the test, probes for which being allergens extracted from allergenic substances derived from natural origins, such as crabs, shrimps, dust mites (Dermatophagoides farinae), house dust mites and storage mites (Blomia tropicalis). Group 10 allergens (tropomyosins) have been assumed to be a major cause of cross-reactivity between house dust mites and other invertebrates.

The sample serum is diluted with diluent buffer containing adequate amount of BSA, no-fat milk, and Tween-20. The peptide array of the present embodiment is incubated with each serum sample respectively at 37° C. for 1 hour. A wash buffer containing Tween-20 is added to the reaction area of the peptide array to wash out extra serum. Anti-human IgE antibody labeled with Cy3 is added to the reaction area of the peptide array. The anti-human IgE with Cy3 binds on antibodies in the sample serum that binds to the peptide probes in the reaction area. The peptide array is then incubated

at 37° C. for 1 hour in darkness. The diluent, wash buffer, and anti-human IgE with Cy3 is provided by EBS Immunoflourescence Specific IgE Assay. A laser scanner with FD532 nm detector is utilized to scan the peptide array after incubation. The intensities of FD532 nm of each peptide probe on the peptide array can be obtained. Results obtained from both the designed peptide arrays and the conventional protein arrays are presented in Table 4. For protein arrays the positive values are highlighted, while for peptide arrays the numbers (No.) of the peptide probes showing positive values are listed. As shown in Table 4, all positive serum samples are positive to dust mites and house dust mites, and partial serum samples also show positive response to other types of antigens. For the peptide array of the present embodiment, although all of the peptide probes are derived from antigens of house dust mites, only a number of probes are bound to the serum samples and generate positive results.

Cross-reactivity usually occurs when an antibody, originally raised against one allergen, binds to a similar allergen from another source. According to the results shown in Table 4, relatively higher reads are observed for protein arrays of dust mites and house dust mites after incubating with serum sample G and W, but none of the probes of the peptide arrays present positive reads. Such results show high risk of false-positive results upon using recombinant proteins as probes. Moreover, the peptide probes of Der P2 on the peptide array almost cover the entire sequence of Der P2, and upon incubation with serum sample B, H, I and Q, only one probe of Der P2 shows positive results. Such results show that although bindings exist between the epitope and IgE, there are no allergic reactions, that is, no cross-linking occurs, thus no histamine can be released from the mast cell. Therefore, the peptide array of the present embodiment could be applied to investigate the influence of distances between epitopes on the cross-linking.

In addition, based on the results shown in Table 4, probes derived from Der P10 present many positive values when incubated with serum samples positive to dust mites, house dust mites, shrimps and crabs (serum samples B, C, D, E, F, K, L, R, S, T, and U). It seems that Der P10 may not be the most allergenic peptide fragment of dust mites, but its' C-terminal: Der p10-16 and Der p10-17 (#59 and #60), may be similar to the allergenic fragments in shrimps and crabs. It can be concluded that when a serum sample presents positive value in the above protein assay to both shrimp and crab antigen. There may be cross-reactivity between Der P10 C-terminal fragments, shrimp antigens and crab antigens, therefore positive values are generated in the test. The positive values are especially significant greater among the serum samples from patients who are allergic to dust mite and house dust mite. In other words, if patient are allergic to dust mite and house dust mite, the allergic syndrome may be intensified due to the consumption of crabs and shrimps.

3. IgE Inhibition Experiments.

Based on the comparisons on tropomyosin peptide sequences in FIG. 10, No. Der-p10-17 peptide fragment (#60) demonstrates homologous sequences in the C-terminal of Por P1 antigen of blue swimmer crab, Pit v1 and Pen a2 antigens of brown shrimp, and peptide probe based on No. Der-p10-17 (#60) peptide fragment can bind to IgE in serum samples. Based on Table 4, the serum sample U is reactive to crab, shrimp, house dust mite, dust mite and Der-p10-17 (#60). Thus, No. Der-p10-17 peptide may be capable to inhibit the binding between IgE in serum sample U and antigens extracted from natural allergic substances in the present embodiment.

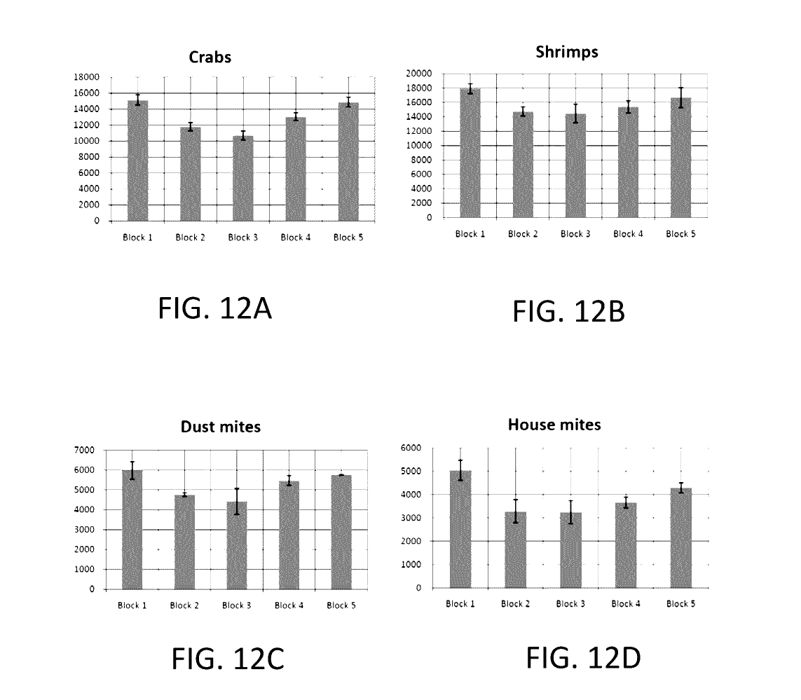

Generally, the serum sample U is incubated with different concentration Der-p10-17 peptide (#60) for 30 minutes, and then serum sample U is transferred on conventional protein arrays spotted with crab, shrimp, dust mite and house dust mite antigens. In each of these arrays, different reaction blocks represent serum sample U incubated with different concentrations of Der-p10-17 (#60), where X1 is serum sample U incubated with the original concentration of Der-p10-17 (#60). X2 is serum sample U incubated with 2-fold dilution of the original concentration, X4 is serum sample U incubated with a 4-fold dilution and X8 is serum sample U incubated with an 8-fold dilution. The experiment protocol in Table 5A-5D and FIGS. 12A-12D may refer to the IgE immunofluorescence assay used in Table 4. The F532 nm intensity value of crabs, shrimps, dust mites, and house mites are shown respectively. Various concentrations of Der-p10-17 peptide (#60) are tested to evaluate the dependence of inhibition on the inhibitor peptide concentration. The results of the IgE inhibition experiments are presented in FIGS. 12A-12D and Tables 5A-5D.

The results of the inhibition experiments demonstrate that No. Der-p10-17 peptide fragment (#60), one fragment of Der-p10 antigen of house dust mites, can inhibit the binding between IgE and antigens extracted from natural house dust mites. IgE in serum sample U have been bind to Der-p10-17 (#60) before incubation on the conventional protein arrays in Table 5A-5D. The remaining free IgE in serum sample U binds on antigens spotted on the conventional protein array. Referring to Table 5A-5D, the larger the value represents the higher amount of free IgE remained after incubated with Der-p10-17 (#60) in the reaction block. As the lesser the concentration of Der-p10-17 (#60), the larger the value. Der-p10-17 (#60) can inhibit the IgE cross-linking between shrimp and crab, the intensity values in presence of No. Der-p10-17 peptide fragment (#60) are also decreased. Therefore, No. Der-p10-17 peptide fragment (#60) is one of the epitopes on Der-p10 antigen specific to IgE, and No. Der-p10-17 peptide fragment (#60) could be used to predict cross-reactivity between antigens, especially as a potential candidate for treat mite, shrimp and crab allergic patient.

TABLE 5A

| | Block 2- | Block 3- | Block 4- | Block 5- | Block 2- |
|---|---|---|---|---|---|
| | X8 | X4 | X2 | X1 | X8 |
| Crabs | 15148 | 11803 | 10695 | 13067 | 14893 |
| STDEV. P | 629 | 503 | 566 | 488 | 595 |
| CV. | 0.04 | 0.04 | 0.05 | 0.04 | 0.04 |

TABLE 5B

| | Block 2- | Block 3- | Block 4- | Block 5- | Block 2- |
|---|---|---|---|---|---|
| | X8 | X4 | X2 | X1 | X8 |
| Shrimps | 17902 | 14759 | 14481 | 15396 | 16678 |
| STDEV. P | 700 | 626 | 1303 | 875 | 1377 |
| CV. | 0.04 | 0.04 | 0.09 | 0.06 | 0.08 |

TABLE 5C

| | Block 2- | Block 3- | Block 4- | Block 5- | Block 2- |
|---|---|---|---|---|---|
| | X8 | X4 | X2 | X1 | X8 |
| Dust mites | 5979 | 4767 | 4428 | 5475 | 5767 |
| STDEV. P | 436 | 100 | 647 | 248 | 11 |
| CV. | 0.07 | 0.02 | 0.15 | 0.05 | 0.00 |

TABLE 5D

| | Block 1- | Block 2- | Block 3- | Block 4- | Block 5- |
|---|---|---|---|---|---|
| | Control | X8 | X4 | X2 | X1 |
| House dust | 5044 | 3288 | 3247 | 3659 | 4288 |
| mites | | | | | |
| STDEV. P | 432 | 496 | 489 | 235 | 211 |
| CV. | 0.09 | 0.15 | 0.15 | 0.06 | 0.05 |

Although the disclosed embodiments only utilize peptide fragments derived from antigens of house dust mites, the above methods of designing peptide fragments and peptide arrays, together with methods of studying cross-reactivity of IgE antibodies to allergens, can be applied to other species of allergens, such as allergens of dust mites, shrimps, crabs, cockroaches, oranges, shrimps, peanuts, pollens or the like.

In assistance of computer-assisted modeling and bioinformatic tools, the present peptide array could be used to screen peptide fragments to develop peptide drugs to inhibit allergic reaction, thus sequences and dosages of peptide drugs could be optimized to inhibit the binding between IgE and antigens. By applying an optimized dosage of one or multiple optimized peptide fragments, degranulation of mast cells process could be efficiently inhibited.

The foregoing descriptions of methods of the present disclosure have been presented for purposes of illustration and description. They are not intended to be exhaustive or to limit the disclosure to the precise methods disclosed and obviously many modifications and variations are possible in light of the above teaching. The examples are chosen and described in order to best explain the principles of the disclosure and its practical application, to enable others skilled in the art to best utilize the disclosure with various modifications as are suited to the particular use contemplated.

What is claimed is:

1. A method for assessing allergen cross-reactivity of at least two species of antigens, using at least one protein analysis device, an epitope database, a processor, and a non-transitory computer readable medium, the method comprising:
analyzing the at least two species of antigens using the at least one protein analysis device, and obtaining species information, amount of each antigen species, and an amino acid sequence of each of the at least two species of antigen;
identifying epitopes of each of the at least two species of antigen using the epitope database by the processor, and obtaining amino acid sequences of the identified epitopes and an amount of each identified epitopes on each antigen species;
comparing the amino acid sequence of each identified epitopes on one of the at least two species of antigens to the amino acid sequence of each of the identified epitopes on the other one of the at least two species of antigens.

2. The method of claim 1, further comprising obtaining an amount of epitopes on one of the at least two species of antigens.

3. The method of claim 1, further comprising obtaining location information of each of the identified epitopes on each of the at least two species of antigens with a bioinformatic software by the processor.

4. The method of claim 4, wherein location information represents distances between every two indentified epitopes on one of the at least two species of antigens.

5. The method of claim 1, further comprising calculating a ratio of the amount of each antigen species in a total amount of the at least two species of antigens by the processor.

6. The method of claim 1, wherein the protein analysis device comprises 2D protein electrophoresis, protein microarrays, high performance liquid chromatography, gas chromatography, or MALDI-TOF mass spectrometry.

7. The method of claim 1, further comprising developing a database of the at least two species of antigens, the database comprises the species information, the amino acid sequence of each of the at least two species of antigen, the amino acid sequences of the identified epitopes, the amount of identified epitopes on each antigen species, and the degrees of similarity.

8. A method for assessing allergen cross-reactivity of at least two species of antigens, comprising:
obtaining amino acid sequence of each of the at least two species of antigens;
identifying epitopes of each of the at least two species of antigen with an epitope database by processing the obtained amino acid sequences, and storing amino acid sequences of the identified epitopes on a non-transitory computer readable medium,
comparing the amino acid sequence of each of the identified epitopes on one of the at least two species of antigen to the amino acid sequence of each of the identified epitopes on the other one of the at least two species of antigen,
providing a peptide library comprising overlapping peptide fragments, amino acid sequences of the peptide fragments are selected from the amino acid sequences of the identified epitopes on one of the at least two species of antigens.

9. The method of claim 8, wherein the amino acid sequences of the at least two species of antigens are obtained from an allergen database.

10. The method of claim 8, wherein overlapping peptide fragments are arrayed on a solid support.

11. The method of claim 8, wherein the at least two species of antigens are allergens of dust mites, house dust mites, shrimps, crabs, cockroaches, oranges, shrimps, peanuts or pollens.

12. A peptide array comprising a solid support surface and a plurality of peptides immobilized on the solid support surface, wherein the plurality of peptides are selected by the following steps:
obtaining an amino acid sequence of an antigen species;
providing a peptide library comprising overlapping peptide fragments with amino acid sequences derived from the obtained amino acid sequence;
comparing amino acid sequences of each of the peptide fragments to amino acid sequences of epitopes in an epitope database;
calculating degrees of similarity of amino acid sequences between each of the overlapping peptide fragments and epitopes in the epitope database by a processor,
selecting the plurality of peptide fragments from the peptide library based on the calculated degrees of similarity of amino acid sequences.

13. The peptide array of claim 12, wherein the plurality of peptides is arrayed on the solid support.

14. The peptide array of claim 12, wherein the antigen species is an allergen.

15. The peptide array of claim 14, wherein the allergen is an allergen of dust mites, house dust mites, shrimps, crabs, cockroaches, oranges, shrimps, peanuts or pollens.

16. The peptide array of claim 14, wherein the overlapping peptide fragments are derived from amino acid sequences of epitopes of the allergen.
